(19) United States
(12) Patent Application Publication
(10) Pub. No.: US 2025/0369023 A1
(43) Pub. Date: Dec. 4, 2025

(54) METHOD FOR CONVERTING CARBON SOURCE INTO ETHYLENE GLYCOL

(71) Applicant: NAN YA PLASTICS CORPORATION, TAIPEI (TW)

(72) Inventors: CHING-YAO YUAN, TAIPEI (TW); CHUNG-YU CHEN, TAIPEI (TW); YI-CHENG LI, TAIPEI (TW); Tzu-Huan Wong, Hualien County (TW); Liang-Jung Chien, TAIPEI (TW)

(21) Appl. No.: 18/792,514

(22) Filed: Aug. 1, 2024

(30) Foreign Application Priority Data
May 29, 2024 (TW) 113119768

(51) Int. Cl. C12P 7/04; C12N 1/20; C12N 9/02; C12N 9/04; C12N 9/06; C12N 9/10; C12N 9/16; C12N 9/88; C12N 15/74; C12N 15/87; C12R 1/01

(52) U.S. Cl. CPC C12P 7/04; C12N 1/20; C12N 9/0006; C12N 9/0008; C12N 9/0022; C12N 9/1096; C12N 9/16; C12N 9/88; C12N 15/74; C12N 15/87; C12R 2001/01; C12Y 101/01095; C12Y 102/01021; C12Y 104/03008; C12Y 206/01052; C12Y 301/03003; C12Y 401/01065

(57) ABSTRACT

A method for converting a carbon source into ethylene glycol includes: providing a plasmid, in which the plasmid includes gene sequences of SEQ ID NO: 1, SEQ ID NO: 2, SEQ ID NO: 3, SEQ ID NO: 4, SEQ ID NO: 5, and SEQ ID NO: 6; implanting the plasmid into cyanobacteria through an electroporation treatment, so that modified cyanobacteria are obtained; and providing the carbon source to the modified cyanobacteria, so that the modified cyanobacteria convert the carbon source into the ethylene glycol.

### CROSS-REFERENCE TO RELATED PATENT APPLICATION

This application claims the benefit of priority to Taiwan Patent Application No. 113119768, filed on May 29, 2024. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a method for converting a carbon source into ethylene glycol, and more particularly to a method for converting a carbon source into ethylene glycol by use of cyanobacteria.

### BACKGROUND OF THE DISCLOSURE

Cyanobacteria are autotrophs that can synthesize the required nutrients by photosynthesis. Since the cyanobacteria have the ability to fix carbon dioxide into metabolites, the cyanobacteria are conventionally applied to production of alcohols and organic acids (e.g., ethanol, butanol, 2,3-butanediol, succinic acid, lactic acid, and isopropylene), so as to reduce the greenhouse effect and damages to the environment. However, there is currently no method that uses one species of cyanobacteria for converting a carbon source into ethylene glycol.

Ethylene glycol (EG) is a diol having the simplest structure, and is often used to manufacture polyester and polyethylene terephthalate (PET). The ethylene glycol is also used in an automobile antifreeze, hydraulic brake fluids, and medical products, and is a valuable chemical. However, the existing ethylene glycol is usually prepared from ethylene oxide (EO), and the ethylene oxide needs to be prepared by silver-catalyzed ethylene oxidation, thereby resulting in a complicated manufacturing process.

Therefore, how to use the cyanobacteria for converting the carbon source into the ethylene glycol through improvements in the manufacturing process, so as to process carbon-containing waste gases and produce valuable chemicals at the same time, has become one of the important issues to be solved in the relevant industry.

### SUMMARY OF THE DISCLOSURE

In response to the above-referenced technical inadequacies, the present disclosure provides a method for converting a carbon source into ethylene glycol.

In order to solve the above-mentioned problems, one of the technical aspects adopted by the present disclosure is to provide a method for converting a carbon source into ethylene glycol. The method includes: providing a plasmid, in which the plasmid includes gene sequences of SEQ ID NO: 1, SEQ ID NO: 2, SEQ ID NO: 3, SEQ ID NO: 4, SEQ ID NO: 5, and SEQ ID NO: 6; implanting the plasmid into cyanobacteria through an electroporation treatment, so that modified cyanobacteria are obtained; and providing the carbon source to the modified cyanobacteria, so that the modified cyanobacteria convert the carbon source into the ethylene glycol.

In one of the possible or preferred embodiments, genomic DNA of the cyanobacteria includes a first locus and a second locus, the gene sequences of SEQ ID NO: 1, SEQ ID NO: 2, and SEQ ID NO: 3 are located at the first locus, and the gene sequences of SEQ ID NO: 4, SEQ ID NO: 5, and SEQ ID NO: 6 are located at the second locus.

In one of the possible or preferred embodiments, the modified cyanobacteria are capable of producing 3-phosphoglycerate dehydrogenase, phosphoserine phosphatase, phosphoserine aminotransferase, serine decarboxylase, ethanolamine oxidase, and glycolaldehyde reductase.

In one of the possible or preferred embodiments, the cyanobacteria are Synechococcus elongatus.

In one of the possible or preferred embodiments, the electroporation treatment is to process for 2 msec to 10 msec at a voltage of between 0.5 kV and 1.5 kV.

In one of the possible or preferred embodiments, the electroporation treatment further includes adding polyethylene glycol having a concentration of between 0.5% and 2%.

In one of the possible or preferred embodiments, the carbon source is carbon dioxide, glucose, sucrose, fructose, or galactose.

In one of the possible or preferred embodiments, the plasmid is an Escherichia coli plasmid.

In one of the possible or preferred embodiments, the method further includes: implanting the plasmid into Escherichia coli for mass production.

In order to solve the above-mentioned problems, another one of the technical aspects adopted by the present disclosure is to provide a method for converting a carbon source into ethylene glycol. The carbon source is converted into the ethylene glycol by using modified cyanobacteria, and the modified cyanobacteria include gene sequences of SEQ ID NO: 1, SEQ ID NO: 2, SEQ ID NO: 3, SEQ ID NO: 4, SEQ ID NO: 5, and SEQ ID NO: 6.

In one of the possible or preferred embodiments, genomic DNA of the modified cyanobacteria includes a first locus and a second locus, the gene sequences of SEQ ID NO: 1, SEQ ID NO: 2, and SEQ ID NO: 3 are located at the first locus, and the gene sequences of SEQ ID NO: 4, SEQ ID NO: 5, and SEQ ID NO: 6 are located at the second locus.

In one of the possible or preferred embodiments, the carbon source is carbon dioxide, glucose, sucrose, fructose, or galactose.

In one of the possible or preferred embodiments, the modified cyanobacteria are capable of producing serine decarboxylase, so as to convert L-serine into ethanolamine.

In one of the possible or preferred embodiments, the modified cyanobacteria are capable of producing ethanolamine oxidase, so as to convert the ethanolamine into glycolaldehyde.

In one of the possible or preferred embodiments, the modified cyanobacteria are capable of producing glycolaldehyde reductase, so as to convert the glycolaldehyde into the ethylene glycol.

Therefore, in the method for converting the carbon source into the ethylene glycol provided by the present disclosure, by virtue of “the plasmid including gene sequences of SEQ ID NO: 1, SEQ ID NO: 2, SEQ ID NO: 3, SEQ ID NO: 4, SEQ ID NO: 5, and SEQ ID NO: 6” and “implanting the plasmid into cyanobacteria through an electroporation treatment, so that modified cyanobacteria are obtained,” the carbon source can be converted into the ethylene glycol by using the modified cyanobacteria. In this way, carbon reduction can be achieved, and valuable chemicals can be obtained at the same time.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a flowchart of a method for converting a carbon source into ethylene glycol according to the present disclosure;

FIG. 2 is a schematic view showing a metabolic pathway of modified cyanobacteria according to the present disclosure;

FIG. 3 and FIG. 4 are each a schematic view showing construction of a plasmid according to the present disclosure;

FIG. 5 is a graph showing calibration curves measured based on different concentrations of the ethylene glycol;

FIG. 6 is a graph showing a measurement result of the ethylene glycol released by cyanobacteria; and

FIG. 7 shows a cell density curve diagram of the modified cyanobacteria cultivated at different temperatures and a bar chart of ethylene glycol production.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

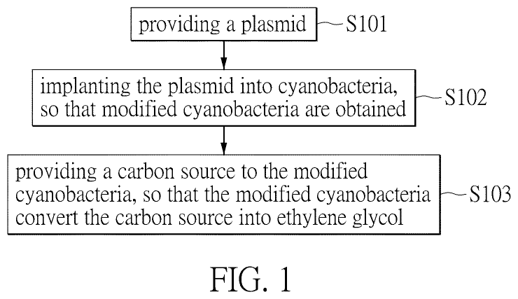

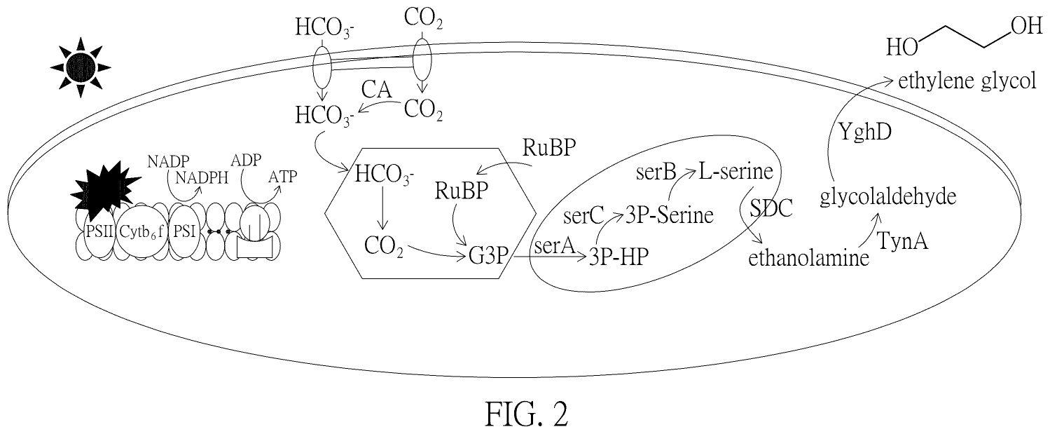

Referring to FIG. 1 and FIG. 2, the present disclosure provides a method for converting a carbon source into ethylene glycol. The method includes: providing a plasmid (step S101); implanting the plasmid into cyanobacteria, so that modified cyanobacteria are obtained (step S102); and providing the carbon source to the modified cyanobacteria, so that the modified cyanobacteria convert the carbon source into the ethylene glycol (step S103). In FIG. 2, NADP refers to nicotinamide adenine dinucleotide phosphate, NADPH refers to reduced nicotinamide adenine dinucleotide phosphate, ATP refers to adenosine triphosphate, ADP refers to adenosine diphosphate, PSII refers to photosystem II, PSI refers to photosystem I, Cytb₆f refers to cytochrome b₆f and is at a center of light-dependent reactions of oxygenic photosynthesis, RuBP refers to ribulose-1,5-bisphosphate, CA refers to carbonic anhydrases, and rbs refers to a ribosome binding site. In addition, SDC refers to serine decarboxylase, TynA refers to ethanolamine oxidase, and YghD refers to glycolaldehyde reductase.

Specifically, in step S101, the plasmid includes gene sequences of SEQ ID NO: 1, SEQ ID NO: 2, SEQ ID NO: 3, SEQ ID NO: 4, SEQ ID NO: 5, and SEQ ID NO: 6. As such, a process of synthesizing a DNA sequence can be further added before step S101. By artificially synthesizing the DNA sequence to be suitable for genetic code recognition of the cyanobacteria, the cyanobacteria can recognize and produce corresponding substances. A genetic code that is suitable for genetic code recognition of Synechococcus elongatus PCC7942 is particularly synthesized in an artificial manner. Furthermore, the plasmid can be an Escherichia coli plasmid, and the plasmid can be implanted into Escherichia coli for mass production.

In step S102, the plasmid is implanted into the cyanobacteria through an electroporation treatment. During the electroporation, cells of the cyanobacteria are subjected to a high voltage and a low capacitance by application of an electric current within an extremely short period of time (ranging from microseconds to milliseconds), so that a potential difference in a cell membrane is formed, and changes occur to the structure of the cell membrane. As a result, the cell membrane is compressed and thinned, thereby generating numerous tiny holes. These tiny holes allow the plasmid to pass through the cell membrane and enter the cells of the cyanobacteria. In one embodiment of the present disclosure, for the electroporation treatment, the cyanobacteria are preferably processed for 10 msec at a voltage of 0.5 kV, are more preferably processed for 5 msec at a voltage of 1.5 kV, and are most preferably processed for 5 msec at a voltage of 1.0 kV, so as to obtain a large colony count.

In order to achieve the optimal plasmid permeability effect, 0.5% to 2% (any concentration ranging between 0.5% and 2%) of polyethylene glycol (PEG) can be further added in this step. For example, the concentration can be 1.0% or 1.5%. Preferably, in the electroporation treatment, the cyanobacteria are processed for 2 msec to 10 msec at any voltage value ranging between 0.5 kV and 1.5 kV (e.g., 0.6 kV, 0.7 kV, 0.8 kV, 0.9 kV, 1.0 kV, 1.1 kV, 1.2 kV, 1.3 kV, and 1.4 kV). The processing time can be any millisecond within a range between 2 msec and 10 msec (e.g., 3 msec, 4 msec, 5 msec, 6 msec, 7 msec, 8 msec, and 9 msec). In the electroporation treatment, when a quantitative concentration of native cyanobacteria is 1×10⁶, amounts of successfully modified cyanobacteria strains under different voltage and time conditions are further tested in the present disclosure (as shown in Table 1 below).

TABLE 1
(1% of PEG added in each group)

| Voltage (kV) | Time (mSec) | Colony Count |
|---|---|---|
| 0.5 | 2 | 6 |
| 0.5 | 5 | 12 |
| 0.5 | 10 | 21 |
| 1.0 | 2 | 19 |
| 1.0 | 5 | 36 |
| 1.0 | 10 | 11 |
| 1.5 | 2 | 17 |
| 1.5 | 5 | 32 |
| 1.5 | 10 | 8 |

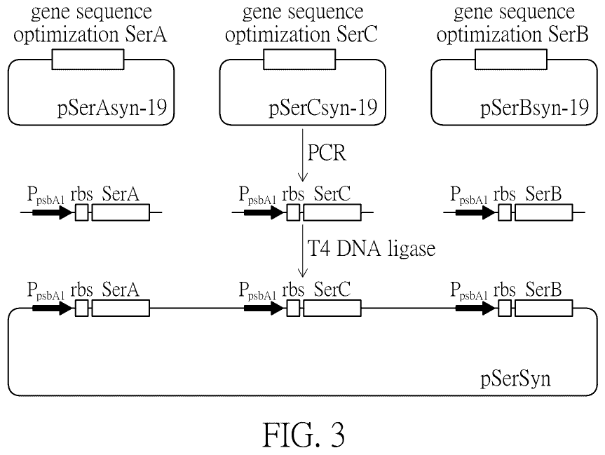

Reference is made to FIG. 3 and FIG. 4. It should be noted that genomic DNA of the cyanobacteria at least includes a first locus and a second locus. In the present disclosure, the gene sequences of SEQ ID NO: 1, SEQ ID NO: 2, and SEQ ID NO: 3 are arranged at the first locus, and the gene sequences of SEQ ID NO: 4, SEQ ID NO: 5, and SEQ ID NO: 6 are arranged at the second locus, so as to increase a gene transfer success rate. Furthermore, a complete chromosome constructed by genetic engineering can be confirmed by being bound to a primer at an NSI locus or an NSII locus.

In one embodiment of the present disclosure, after the gene sequences of SEQ ID NO: 4, SEQ ID NO: 5, and SEQ ID NO: 6 are arranged at the second locus, the gene sequences of SEQ ID NO: 1, SEQ ID NO: 2, and SEQ ID NO: 3 are arranged at the first locus by the same transfer strategy. In other words, by using different genomes for a homologous crossover, the locus of SEQ ID NO: 1, SEQ ID NO: 2, and SEQ ID NO: 3 and that of SEQ ID NO: 4, SEQ ID NO: 5, and SEQ ID NO: 6 can be different. In another embodiment of the present disclosure, after the gene sequences of SEQ ID NO: 1, SEQ ID NO: 2, and SEQ ID NO: 3 are arranged at the first locus, the gene sequences of SEQ ID NO: 4, SEQ ID NO: 5, and SEQ ID NO: 6 are arranged at the second locus by the same transfer strategy. That is to say, as long as the gene sequences can be correspondingly arranged at the first locus and the second locus, there is no specific limitation on an arrangement order of the gene sequences.

After modified plasmid DNA is sequentially transferred to the native cyanobacteria, antibiotics (in an order of spectinomycin and kanamycin) can be used to filter out the modified cyanobacteria that have obtained SEQ ID NO: 1, SEQ ID NO: 2, SEQ ID NO: 3, SEQ ID NO: 4, SEQ ID NO: 5, and SEQ ID NO: 6 after a successful homologous crossover. In detail, filtering is performed by use of the spectinomycin and the kanamycin, and the successfully modified cyanobacteria strains can grow on a solid medium (BG-11) containing the antibiotics. More specifically, since the gene sequences of SEQ ID NO: 1, SEQ ID NO: 2, and SEQ ID NO: 3 are arranged at the first locus, and the gene sequences of SEQ ID NO: 4, SEQ ID NO: 5, and SEQ ID NO: 6 are arranged at the second locus, the gene transfer success rate can be increased in the present disclosure.

In one embodiment of the present disclosure, SEQ ID NO: 1 can be a gene encoding ethanolamine oxidase (Tyna), or a gene having Tyna activity and at least 80% sequence identity to SEQ ID NO: 1. SEQ ID NO: 2 can be a gene encoding glycolaldehyde reductase (YghD), or a gene having YghD activity and at least 80% sequence identity to SEQ ID NO: 2. SEQ ID NO: 3 can be a gene encoding serine decarboxylase (SDC), or a gene having SDC activity and at least 80% sequence identity to SEQ ID NO: 3. SEQ ID NO: 4 can be a gene encoding 3-phosphoglycerate dehydrogenase (SerA), or a gene having SerA activity and at least 80% sequence identity to SEQ ID NO: 4. SEQ ID NO: 5 can be a gene encoding phosphoserine phosphatase (SerB), or a gene having SerB activity and at least 80% sequence identity to SEQ ID NO: 5. SEQ ID NO: 6 can be a gene encoding phosphoserine aminotransferase (SerC), or a gene having SerC activity and at least 80% sequence identity to SEQ ID NO: 6.

Firstly, 10 μg/mL of the spectinomycin is used to filter out cyanobacteria that are capable of producing 3-phosphoglycerate dehydrogenase (SerA), phosphoserine phosphatase (SerB), and phosphoserine aminotransferase (SerC). Then, 5 μg/mL to 10 μg/mL of the kanamycin is used to filter out cyanobacteria that are capable of producing serine decarboxylase (SDC), ethanolamine oxidase (TynA), and glycolaldehyde reductase (YghD).

After the successful homologous crossover, the modified cyanobacteria are capable of simultaneously producing the 3-phosphoglycerate dehydrogenase (SerA), the phosphoserine phosphatase (SerB), the phosphoserine aminotransferase (SerC), the serine decarboxylase (SDC), the ethanolamine oxidase (TynA), and the glycolaldehyde reductase (YghD). In this way, the ability to use one species of the cyanobacteria for converting the carbon source into the ethylene glycol and releasing the same outside of the cells can be obtained, and the ethylene glycol can be obtained without cell disruption.

Specifically, the modified cyanobacteria of the present disclosure still retain the property of the native cyanobacteria to convert the carbon source into glyceraldehyde 3-phosphate (G3P). In addition, the modified cyanobacteria of the present disclosure are capable of producing the 3-phosphoglycerate dehydrogenase (SerA), so as to convert the glyceraldehyde 3-phosphate (G3P) into 3-phosphohydroxypyruvate (3P-HP). The modified cyanobacteria of the present disclosure are also capable of producing the phosphoserine aminotransferase (SerC), so as to convert the 3-phosphohydroxypyruvate (3P-HP) into 3-phosphoserine (3P-serine). The modified cyanobacteria of the present disclosure are further capable of producing the phosphoserine phosphatase (SerB), so as to convert the 3-phosphoserine (3P-serine) into serine (especially L-serine).

Afterwards, since the modified cyanobacteria of the present disclosure are capable of producing the serine decarboxylase, the L-serine can be converted into ethanolamine. The ethanolamine oxidase produced by the modified cyanobacteria of the present disclosure can be further used to convert the ethanolamine into glycolaldehyde. Lastly, the modified cyanobacteria of the present disclosure are capable of producing the glycolaldehyde reductase, so as to convert the glycolaldehyde into the ethylene glycol.

In one embodiment of the present disclosure, in order to convert the carbon source into the ethylene glycol by use of the modified cyanobacteria, cultivation is conducted under the following conditions: a temperature of 42° C., a luminous intensity of 200 μmol/m⁻²/s⁻¹, a 12-hour light/12-hour dark cycle, an initial use of 25 mM of sodium bicarbonate (NaHCO₃), and a carbon dioxide concentration of 3%. After 72 hours of cultivation in a jar test, 1,025 mg/L of ethylene glycol can be produced.

In the present disclosure, the carbon source can be an industrial waste gas (i.e., a mixture of hydrogen, acetylene, methane, hydrogen sulfide, and acetaldehyde). Specifically, the mixture can include 30 ppm to 50 ppm of the hydrogen, 150 ppm to 250 ppm of the acetylene, 100 ppm to 200 ppm of the methane, 0.1 ppm to 1 ppm of the hydrogen sulfide, and 1 ppm to 5 ppm of the acetaldehyde. For example, the industrial gas can be a mixture of 40 ppm of the hydrogen (H₂), 200 ppm of the acetylene (C₂H₂), 150 ppm of the methane (CH₄), 0.5 ppm of the hydrogen sulfide (H₂S), and 3 ppm of the acetaldehyde (CH₃CHO).

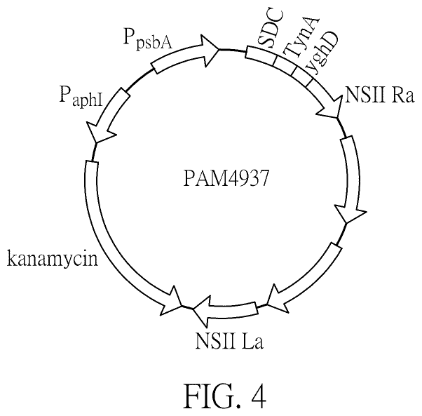

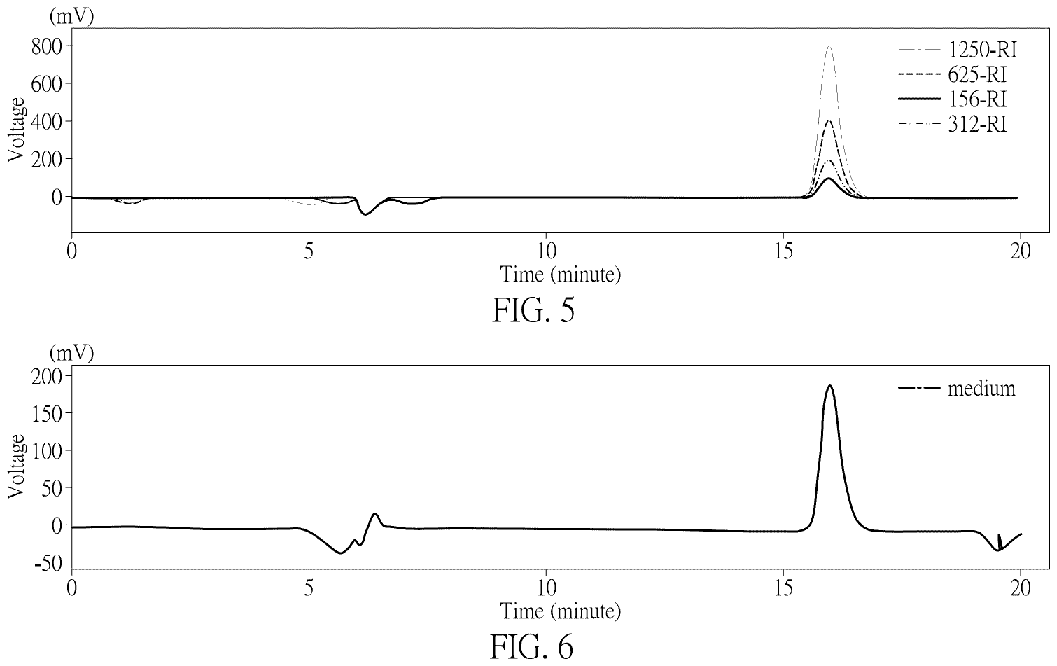

Referring to FIG. 5 and FIG. 6, FIG. 5 is a graph showing calibration curves measured based on different concentrations of the ethylene glycol, and FIG. 6 is a graph showing a measurement result of the ethylene glycol released by the cyanobacteria. In FIG. 5, a refractive index detector is used for analysis and detection, and a graph in which concentrations of the ethylene glycol are respectively 156 mg/mL, 312 mg/mL, 625 mg/mL, and 1,250 mg/mL is shown. In one embodiment of the present disclosure, the modified cyanobacteria of the present disclosure are cultivated at a temperature of 37° C. and a carbon dioxide concentration of 3% for 60 hours. Then, such a medium is directly analyzed, and results thereof are as shown in FIG. 6. That is to say, after the modified cyanobacteria of the present disclosure are cultivated, the concentration of the ethylene glycol directly detected in the medium is approximately 1,025 mg/mL. In other words, the modified cyanobacteria of the present disclosure can indeed convert the carbon source into the ethylene glycol, and directly release the same outside of strains.

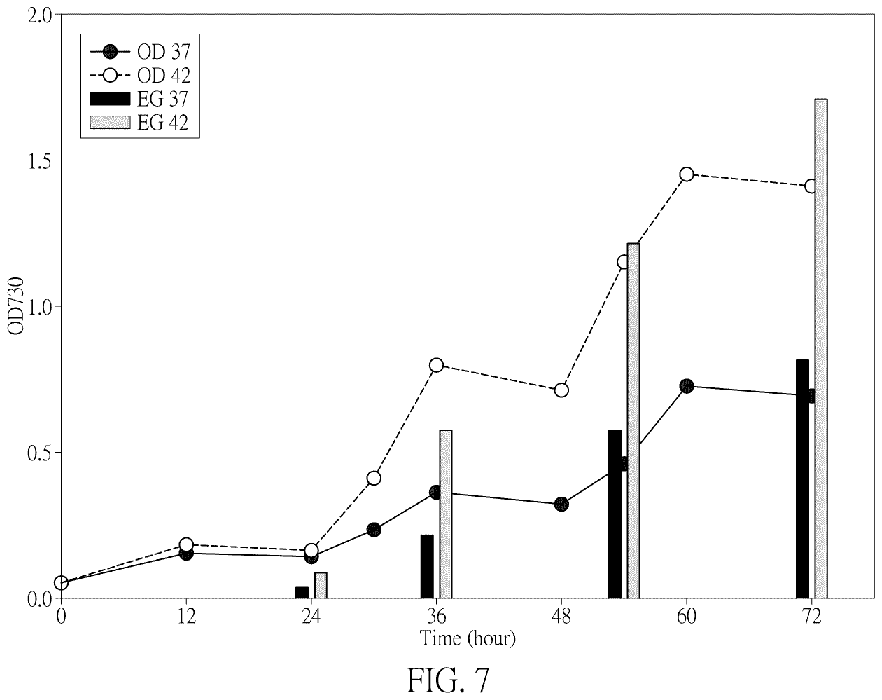

Moreover, the modified cyanobacteria of the present disclosure are suitable to grow in an environment of between 30° C. and 60° C., so as to be used for processing the industrial waste gas. Preferably, the modified cyanobacteria of the present disclosure are suitable to grow in an environment of between 35° C. and 50° C. In one embodiment of the present disclosure, without changing the remaining cultivation conditions, the modified cyanobacteria of the present disclosure are cultivated at a temperature of 37° C. and 42° C., and a growth status of the modified cyanobacteria and an accumulation amount of the ethylene glycol are measured by optical density (OD₇₃₀). In FIG. 7, OD37 represents a cell density curve of the modified cyanobacteria at a cultivation temperature of 37° C., OD42 represents a cell density curve of the modified cyanobacteria at a cultivation temperature of 42° C., EG37 represents the accumulation amount of the ethylene glycol (in the form of a bar chart of ethylene glycol production) at the cultivation temperature of 37° C., and EG42 represents the accumulation amount of the ethylene glycol (in the form of a bar chart of ethylene glycol production) at the cultivation temperature of 42° C. A growth density of the modified cyanobacteria and the accumulation amount of the ethylene glycol increase with cultivation time. It should be noted that, by cultivating the modified cyanobacteria at the temperature of 42° C. (as compared with cultivating the modified cyanobacteria at the temperature of 37° C.), a growth speed of the modified cyanobacteria can be increased, and a higher production amount of the ethylene glycol can be obtained. In other words, the modified cyanobacteria of the present disclosure are particularly suitable to grow in an environment of 42° C., which is beneficial for being applied to processing of the industrial waste gas.

### BENEFICIAL EFFECTS OF THE EMBODIMENT

In conclusion, in the method for converting the carbon source into the ethylene glycol provided by the present disclosure, by virtue of “the plasmid including gene sequences of SEQ ID NO: 1, SEQ ID NO: 2, SEQ ID NO: 3, SEQ ID NO: 4, SEQ ID NO: 5, and SEQ ID NO: 6” and “implanting the plasmid into cyanobacteria through an electroporation treatment, so that modified cyanobacteria are obtained,” the carbon source can be converted into the ethylene glycol by using the modified cyanobacteria. In this way, carbon reduction can be achieved, and valuable chemicals can be obtained at the same time.

Furthermore, in the present disclosure, by virtue of “the gene sequences of SEQ ID NO: 1, SEQ ID NO: 2, and SEQ ID NO: 3 being located at the first locus, and the gene sequences of SEQ ID NO: 4, SEQ ID NO: 5, and SEQ ID NO: 6 being located at the second locus,” the gene transfer success rate can be increased.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A method for converting a carbon source into ethylene glycol, comprising:
providing a plasmid, wherein the plasmid includes gene sequences of SEQ ID NO: 1, SEQ ID NO: 2, SEQ ID NO: 3, SEQ ID NO: 4, SEQ ID NO: 5, and SEQ ID NO: 6;
implanting the plasmid into cyanobacteria through an electroporation treatment, so that modified cyanobacteria are obtained; and
providing the carbon source to the modified cyanobacteria, so that the modified cyanobacteria convert the carbon source into the ethylene glycol.

2. The method according to claim 1, wherein genomic DNA of the cyanobacteria includes a first locus and a second locus, the gene sequences of SEQ ID NO: 1, SEQ ID NO: 2, and SEQ ID NO: 3 are located at the first locus, and the gene sequences of SEQ ID NO: 4, SEQ ID NO: 5, and SEQ ID NO: 6 are located at the second locus.

3. The method according to claim 1, wherein the modified cyanobacteria are capable of producing 3-phosphoglycerate dehydrogenase, phosphoserine phosphatase, phosphoserine aminotransferase, serine decarboxylase, ethanolamine oxidase, and glycolaldehyde reductase.

4. The method according to claim 1, wherein the cyanobacteria are Synechococcus elongatus.

5. The method according to claim 1, wherein the electroporation treatment is to process for 2 msec to 10 msec at a voltage of between 0.5 kV and 1.5 kV.

6. The method according to claim 1, wherein the electroporation treatment further includes adding polyethylene glycol having a concentration of between 0.5% and 2%.

7. The method according to claim 1, wherein the carbon source is carbon dioxide, glucose, sucrose, fructose, or galactose.

8. The method according to claim 1, wherein the plasmid is an Escherichia coli plasmid.

9. The method according to claim 1, further comprising:
implanting the plasmid into Escherichia coli for mass production.

10. A method for converting a carbon source into ethylene glycol, characterized in that the carbon source is converted into the ethylene glycol by using modified cyanobacteria, and the modified cyanobacteria include gene sequences of SEQ ID NO: 1, SEQ ID NO: 2, SEQ ID NO: 3, SEQ ID NO: 4, SEQ ID NO: 5, and SEQ ID NO: 6.

11. The method according to claim 10, wherein genomic DNA of the modified cyanobacteria includes a first locus and a second locus, the gene sequences of SEQ ID NO: 1, SEQ ID NO: 2, and SEQ ID NO: 3 are located at the first locus, and the gene sequences of SEQ ID NO: 4, SEQ ID NO: 5, and SEQ ID NO: 6 are located at the second locus.

12. The method according to claim 10, wherein the carbon source is carbon dioxide, glucose, sucrose, fructose, or galactose.

13. The method according to claim 10, wherein the modified cyanobacteria are capable of producing serine decarboxylase, so as to convert L-serine into ethanolamine.

14. The method according to claim 13, wherein the modified cyanobacteria are capable of producing ethanolamine oxidase, so as to convert the ethanolamine into glycolaldehyde.

15. The method according to claim 14, wherein the modified cyanobacteria are capable of producing glycolaldehyde reductase, so as to convert the glycolaldehyde into the ethylene glycol.


SEQUENCE LISTING (6 sequences)

SEQ ID NO: 1 - DNA, 2274 bp, Synechococcus elongatus
  source 1..2274: mol_type = genomic DNA
atgggttcgcccagcctctactcagctcgaaagacgacgttggcacttgctgttgcgctctcctttgcctggcaggcaccagtgtttgctcatggtggcgaagcccacatggtcccgatggataaaactctcaaagaatttggcgcagacgttcagtgggatgactatgcccaactgttcacactgattaaggatggagcctatgtcaaagtcaagccaggggcgcaaacggcgatcgtcaacggccagccgctggccttgcaagttcctgttgtgatgaaggataacaaagcctgggtgagcgatacctttatcaacgatgtcttccagtcgggtctcgaccagacttttcaagtggaaaaacggccccatccactgaatgcgctgaccgccgatgagatcaaacaagccgtcgagattgtgaaagcgagtgctgacttcaaaccaaacacccgctttaccgaaatcagcttgctgccacccgacaaagaggcagtttgggcctttgcactggagaacaagcccgtcgatcagccccgcaaggccgatgtgattatgctggatggtaagcatatcatcgaagctgtcgtcgatctgcaaaacaacaaactgctaagttggcaaccgatcaaggatgctcacggcatggtgcttttggatgattttgccagcgtgcagaatattatcaataacagcgaggagtttgccgctgctgtcaagaagagaggcatcacggatgcaaagaaagtgatcaccacgccgttgactgtgggctacttcgatggcaaagatggactgaagcaggatgcgcggttactgaaggtcatatcatatctcgatgtgggggatggtaactactgggcgcatccgatcgaaaatcttgttgccgtcgtggatctggaacaaaaaaagattgtgaaaatcgaggaaggaccggtcgtgccagttccgatgacagcgcgtccctttgatggccgcgatcgcgtcgcaccggcagtcaagcccatgcaaattattgagccggagggcaaaaactatacgattactggtgacatgattcactggcgcaactgggactttcatttgtcgatgaacagccgggtcggcccaatgatctccacggtgacctacaatgacaatggcaccaagcgcaaggtcatgtacgaaggcagcttggggggcatgatcgtaccctatggcgatcctgatattggctggtatttcaaggcctacctagactctggcgattatggtatgggcaccttgacgtcccctattgcccgtggcaaagatgcccccagtaatgccgtgctgctcaatgaaacgatcgctgactacaccggcgtaccgatggaaattccacgcgcgatcgcggtttttgagcgctacgccggaccagaatacaaacaccaagaaatggggcaacccaatgtcagtacagaacgccgcgaactggtggttcgctggatttctactgttggcaactacgactacatctttgactggattttccacgaaaatggaacgattggtattgacgcaggtgccacgggcattgaagcggtcaaaggggtgaaagcaaaaaccatgcatgacgagacagctaaagacgatacacgatacggaacacttatcgatcacaacatcgtgggcacgacccatcagcacatttacaacttccgtctcgatttggacgtggacggtgagaataatagtctggtggcgatggaccccgttgttaagcctaacactgcgggggggccacggaccagcacgatgcaggtcaatcaatataacattgggaatgagcaagatgcggcgcagaaatttgatccggggaccattcgcctattaagcaacccgaataaggaaaatcgcatgggcaatcctgtaagctatcagatcattccctacgctggtggtactcatcctgtagccaagggcgcgcagtttgcccccgatgaatggatctatcaccgtctgtccttcatggataagcaactatgggtcacccgctaccaccccggtgaacgattcccggaagggaaatatcccaaccgatcgacccacgatacgggactcggtcagtacagcaaagacaacgaaagtctcgacaacaccgatgccgttgtctggatgacgaccggtacaactcatgtcgctcgagctgaagagtggcccatcatgcctactgagtgggttcataccctactcaagccttggaatttcttcgatgaaaccccgaccttaggcgcgttgaaaaaagacaagtaa

SEQ ID NO: 2 - DNA, 1164 bp, Synechococcus elongatus
  source 1..1164: mol_type = genomic DNA
atgaacaacttcaacctgcatacaccgacccgtattctgtttgggaaaggagcaatcgcggggctccgcgagcaaattccccacgatgctcgggtgctcatcacctacggcgggggctccgtcaagaaaaccggagttttagatcaggtacttgatgctctcaaagggatggatgtcctagaatttggcggcatcgaacccaatcctgcctacgaaaccttgatgaatgccgtcaagctggttcgcgaacagaaagtgaccttcctcctggcagtgggcggtggttccgtcttggatggtactaagtttattgctgcggcggctaactatcccgaaaacatcgatccgtggcatatcttgcagactggcggcaaggaaatcaaatcggcgatcccgatgggctgcgtgctgacactcccggctacgggttcggaaagcaacgcgggcgccgtgataagccgcaagacgacgggcgataaacaggcgttccacagcgctcatgtgcagcccgtttttgcggtgttggacccagtctacacctatactttacctccccgccaagtggccaatggcgtggttgatgcctttgtccacaccgttgagcagtacgtcacaaagcccgtcgatgccaagattcaagatcgcttcgccgaaggcattttgctaacgctgattgaagatgggcccaaagcgctgaaggagccggagaactacgacgtgcgagccaatgtgatgtgggcagcaacgcaagcgctgaatggtcttattggtgcaggtgtcccgcaggactgggctacccacatgctagggcatgagctgactgccatgcacggtctggaccatgcccaaaccctggcgatcgtcttgccagccctctggaatgaaaaacgcgatacgaagcgcgccaaactgctgcaatatgccgagcgggtttggaacattacagagggcagcgatgatgagcgtatcgacgcggcgatcgctgctacaagaaatttctttgaacaattgggagtaccaactcatttaagtgactatggtcttgatggcagttcaattcctgctttgctcaagaaactcgaagagcacggcatgacccagctcggggaaaatcacgacattacgctcgacgtttctcgacggatctacgaggcagcacgttaa

SEQ ID NO: 3 - DNA, 1449 bp, Synechococcus elongatus
  source 1..1449: mol_type = genomic DNA
atggtcggtagtctcgaaagtgatcaaaccctgagtatggctacgctgattgaaaaactggacatcctttccgacgatttcgatccgaccgctgttgtgactgaaccattgccaccccctgtaactaatgggattggcgcagataagggtggcggtggaggcgagcgtgaaatggtgctaggccgcaatattcacacgacgtcccttgcagtcaccgagccagaggttaatgatgagtttaccggtgacaaggaagcctacatggcaagtgtattggcacggtatcgcaaaaccttggtggaacggactaaaaatcacttgggttatccctacaacttagactttgactatggcgcgttgggtcagctccagcatttctcgatcaacaacctgggagatcccttcattgaatcgaactacggcgtgcacagccgtccctttgaggtcggtgttttggattggtttgcccgtctgtgggagattgagagagacgattactggggctatattaccaattgtggcacagaaggcaatctgcatggaatcttggttggacgggagatgtttccggatggcatcctctacgccagccgggagtcccactactcggtcttcaaagccgcgcgaatgtatcgaatggaatgcgaaaaagttgacacgctgatgtctggggaaatcgattgtgatgacctgcgcaaaaagctgctcgcgaacaaggacaagcctgccattctcaacgtcaacattgggaccactgtcaaaggggctgtggatgatctcgatctggttattaaaacgctcgaagagtgcggctttagccacgatcgcttctacatccattgcgatggtgccctctttggcctaatgatgccgtttgtcaagcgcgcccccaaagtcaccttcaacaaaccgattggaagtgtctctgtctctgggcacaaattcgtgggctgtccgatgccctgtggggttcaaatcacgcggatggagcatattaaggtgcttagcagcaatgtcgagtatttagcgagccgcgatgcgaccatcatgggctcgcggaatggccatgccccgctgttcctctggtacaccctaaatcgcaagggttacaaaggcttccaaaaagaagtgcagaaatgtttacgtaacgctcattacctgaaagatcgcctccgagaggctggtatttcagcgatgctgaacgaactctccagcacggtggtgtttgaacgcccgaaggacgaagaatttgtgcgccgctggcaactggcttgccagggcgacatcgcgcatgtggtagtgatgccttcagttacaatcgagaagttggataactttctgaaggatctagttaagcaccgcctgatctggtatgaagatgggtcgcagcccccctgcctagcaagcgaagttggtacaaataattgcatctgccctgcccacaagtaa

SEQ ID NO: 4 - DNA, 1590 bp, Synechococcus elongatus
  source 1..1590: mol_type = genomic DNA
atgcccaaggtcttggtgtccgatccaatcgatcaggttggtcttgatatcctctcgcaagtggcccaagtggatgtcaaaaccggcctgagtcctagtgagctagcgcaaatcataggtgaatatgacgccctgatgctgcgcagcgggacacgtgtcacggccgaagtgatcgaagccggccaaaaactgcgtattattggtcgggctggggttggtgttgacaatgttgatgtcccggcggctacccgccggggaattgtggtcgtcaatagcccagaaggcaatactatcgccgcggcagagcacacactcgcaatgatgttgtcgctgagccgccacattccggatgccaatgcttccactaagagtggtggctgggatcgcaagtcttttgtcggcactgaggtctacaaaaaaaccctgggggttgttggtcttggcaaaatcggaagccacgtcgcgacggtcgcgaaggcgatgggcatgaaactcctggcctacgacccctttatttcagcagaacgagcggaacagattggggcccggttggtcgagttggatattctctttcaggaagcagactacatcacgctgcatattcccaaaacccccgaaaccgccaacctgatcaatgctgaaaccctagccaagatgaagcccaccactcgcattatcaactgcgcccgtggaggggtcatcaacgaacaagcacttgcggatgcgatcgctgctggcaaaatcggcggtgcggccctggatgtttacgatcaagagcccctccaggctgattcaccgctgcgagcactgggtaagaacctgatcttgacgccccatctaggtgcctccacaaccgaggcacaggtcaacgtcgcagtagacgtagcggagcagattcgggacgtcctcctgggcctcccagcccggagtgccgtgaacatccctgggctctatcctgatattttggaaaagctgcggccgtatctgcagttggcggaaactctgggcaacctccttagtcaagtcgcgggcggccgcctcgagcaactcacggttcgattgcaaggcgagttggctgcccagcaatctcagccgatcgtggtggctgctttaaaaggcctattgacccaagcattacgagaacgtgttaactatgtcaatgctatgatcgaggccaaagagcgcggaattcgcatcatcgaaacacgcgatgagagcgtgcgcgatgactatgccggggggtccttgcagctggttgccacgggcagcaatggtgaacatcgtgtgatgggcgctctgttgggcgatggcgagattcggattaccaatgtggacgaattccctgtgaacgtcccgcccagccgatacatgctgttcacgcggcaccgcgacatgccgggcatcattggaaaaattgggtcgctcttgggttctttcaacgtgaatattgcctcgatgcaggttggacgccgcattgtgcgcggtgacgcggtgatggtgctgtcgctcgatgatccgttaccagaaggaatcctagcggaaattacgaaggtggctggcatcagcgatgcttacaccgttgtgctctaa

SEQ ID NO: 5 - DNA, 1338 bp, Synechococcus elongatus
  source 1..1338: mol_type = genomic DNA
atggcaacacgtgtcgtcttagtgcgtcatgggcaaagcagctacaacgccgctggtcggatccaaggacgttgcgataactcgcaactgactgatcgaggcgcagcggacgcagtcaaagttgcagctgcactgaatggcattcccttcgctgctgcctactgctcaccactgcagcgagccaaacggacggcagaaatcatcattgagcagatcgaaacgccgccagctttggcggtgagcgatggccttctcgaagtcgacctgccgctgtgggagggcctcagtcgcgaagaagtgcgctctcagtacgccgaactctatcgccagtggcacgaagccccccatgagctagtcctgaccgtgccggacggtcaaggtggcagccgcgagcatgcccctgttttggctctgtttgagcaagcgcggcagttttggaaggatttgttggaacggcatcgcgatcaaactgtactactggttgcccacaacgggattctgcgctcactgatcgcgacggcattgggcgttgatcctagcgcctatcaagtgatccgacaatctaactgcgggatcagtgtgctcaacttcgcagatggaaccaatcagccggcccagctggaatgtctaaatctaaccgcaccgcttggcgatgccctccctgatcgcggcgcttcgtcgggcgtccgacttctgctggttcgccatggtgaaaccgactggaatcgccaaaaacgattccaagggcaaatcgatattccgctgaatgacaacggtagggctcaggcgcgtagtgcggccgaatttttggcgcccattcagattgactttgcggtctcctcgccgatggcgcggccgaaggaaacggctgagctgattttggagcgccaccccaattgtgaactgtcggtggacgatcgcctccaagaaattggtcacggcctctgggagggaaagctcgaggaagagatcgcagcggaattcggcgaattgttacagctctggaaagatcagcccgaacaggtgcagatgcccgagggggagaacctgcaagaggtttgggatcgcagcgtcgcggcttgggaagcgatcgtcgccaatgccccagaaggcagtaccggcttagtggtcgctcacgatgcggtcaacaaagtgattctatgccacgtcttgggcttgtcccccgccgacatttggagcatcaagcagggcaacggggctgttacagtggttgactatcccaagcggctcgattcccgtccagtactccaagccatgaatctgactctgcatctcgatggtgccgtgttggatcgcaccgccgcgggtgctctttaa

SEQ ID NO: 6 - DNA, 1080 bp, Synechococcus elongatus
  source 1..1080: mol_type = genomic DNA
atgatgggcaatcgcgtatacaacttcaacgcaggaccttctgcgatgccgctgcccgtgttagaggaaattcagcaggagtttctagactttaatcacacgggcatgagcatcgtggaaatcagccatcgatcggatgcatatcaaaagatgcaggatgaaacgattgcgctccttcataagttattgaatattcccgacgagtacggcattaccttcatgcaaggcggtggttcgatgcagttcctgatgcatggcgctaattttttgaagcaccgcgggggctacatcaatactggtgtctggagtaaaaaagccaaagagggtgcggctttttatggcgatgtctacgacgttgctacgagtgagggcgaaggttttcggcggattccgggcccagaggaattcgatctgctgcctgataccgactacgtctatctgaccagcaacaacactattcacggcacacagtggcaagccttcccccacatcgatctcccgctaatatgcgatatgtcgagtgattttctttcgcggcctgtggatgtgaagaactttgacttcatctatgctgggattcaaaaaaatgttggaccagcgggggcagtgattggtatcgcaaagcattcatacctggaaacagcccgtcaagacctgccgatcatgctgcagtacaaaaccttttttgatcacgacagtacttataacacgccgcccgttttttgcatctacttcgtcaacaaggtgttgcactggctcgatgagcgaggggggctcaaggagactgagcggatcaacaaagaaaaggcggcgtttgtctacgatgcgatcgatgccagcgatggcttctatcgcccccatgccgccaaggattcacgcagcttgatgaacatcaccttcaatctggtgacgccagaactcgagaaagaatttgtttcccgtgcagccgaagaaaaactgattggtctcaaaggtcatcgttctattggcggctgtcgcgcttccacctataatgctgtcaccaaagaagcctgtgaacgcttggccgactttatgcaccgcttccgcaaagttcattaa